(19) United States
(12) Patent Application Publication
(10) Pub. No.: US 2010/0286155 A1
(43) Pub. Date: Nov. 11, 2010

(54) ADRENOCORTICAL CARCINOMA TREATMENT

(75) Inventors: Elizabeth A. BUCK, Huntington, NY (US); Andrew W. STEPHENS, Boulder, CO (US)

Correspondence Address: OSI PHARMACEUTICALS, INC., 420 Saw Mill River Road, Ardsley, NY, 10502, US

(73) Assignee: OSI PHARMACEUTICALS, INC., Ardsley, NY (US)

(21) Appl. No.: 12/775,556

(22) Filed: May 7, 2010

Related U.S. Application Data
(60) Provisional application No. 61/180,249, filed on May 21, 2009.
(60) Provisional application No. 61/176,346, filed on May 7, 2009.

(51) Int. Cl. A61K 31/4985; A61P 35/00

(52) U.S. Cl. 514/249

(57) ABSTRACT

A method of treating adrenocortical carcinoma with OSI-906.

### FIELD AND BACKGROUND

This application claims priority of U.S. Appl. Ser. Nos. 61/176346 (filed 7 May 2009) and 61/180249 (filed 21 May 2009), which are incorporated herein by reference in their entireties.

The present invention pertains in some aspects to cancer treatment, adrenocortical carcinoma or adrenal cortex cancer (ACC), small molecule molecular targeted therapies, and IGF-1R inhibitors.

ACC is a rare endocrine malignancy having a generally poor prognosis. Treatments for ACC include surgery, radiation, chemotherapy, hormone treatment, and mitotane. See J. Clin. Endocrinol. Metab., 91, 2027-2037 (2006); Cancer, 113, 11, 3130-3136 (2008).

Logié et al. state that the IGF system is involved in ACC and that the NCI H295R cell line, which is derived from a human adult ACC, is a suitable in vitro model for studying the molecular mechanisms of ACC tumor proliferation. J. Molec. Endocrinol., 23, 23-32 (1999); see also Rev. Endocr. Metab. Disord., 8, 343-348 (2007).

Hammer et al. state that insulin-like growth factor 2 (IGF2) is the most upregulated transcript in 80-90% of ACCs, and that IGF pathway signaling plays a critical role in ACC pathophysiology. J. Clin. Endocrinol. Metab., doi:10.1210/jc.2008-1456; see also J. Clin. Endocrinol. Metab., doi:10.1210/jc.2008-0065.

Giordano et al. describe molecular profiling of ACC tumors that revealed two subtypes showing significantly different survival rates. Clin. Cancer. Res., 15(2), 668-676 (2009).

OSI-906 is a small molecule IGF-1R inhibitor disclosed in U.S. 2006/0235031, Example 31. As of 2009, OSI-906 is in development by OSI Pharmaceuticals, Inc. Clinical efficacy of OSI-906 in ACC has been documented.

There is need for new ACC therapies, including small molecule oral therapies such as IGF-1R inhibitors.

### SUMMARY

In some aspects, the present invention provides a method of treating adrenocortical carcinoma (ACC) with OSI-906.

### DRAWINGS

FIG. 1. ACC Patient 1 primary adrenal mass baseline CT scan (21 Jul. 2008);

FIG. 2. ACC Patient 1 primary adrenal mass CT scan (9 Mar. 2009);

FIG. 3. ACC Patient 1 right lower lobe metastasis baseline CT scan (21 Jul. 2008);

FIG. 4. ACC Patient 1 right lower lobe metastasis CT scan (9 Mar. 2009);

FIG. 5. ACC Patient 1 pulmonary metastasis baseline CT scan (21 Jul. 2008);

FIG. 6. ACC Patient 1 pulmonary metastasis CT scan (9 Mar. 2009);

FIG. 7. ACC Patient 1 pulmonary metastasis baseline CT scan (21 Jul. 2008);

FIG. 8. ACC Patient 1 pulmonary metastasis CT scan (9 Mar. 2009);

FIG. 9. ACC Patient 1 RECIST data;

Fig. 10. ACC Patent 1 FOG-PET scan 7 Apr. 2009;

FIG. 11. H295R ACC tumor cell line OSI-906 sensitivity;

FIG. 12. pIGF-1R and pIR 051-906 inhibition.

### DETAILED DESCRIPTION

### Patients

According to the present invention, the patient selected for treatment is suffering from ACC.

In some embodiments, the patient exhibits a histologically or cytologically documented malignancy that is advanced, metastatic, or refractory to established forms of therapy or for which no effective therapy exists.

In some embodiments, the selected patient had prior discontinued chemotherapy, radiation, surgery, or hormonal therapy.

In some embodiments, the ACC is refractory to prior mitotane-containing treatment.

In some embodiments, the ACC is not previously treated.

In some embodiments, the selected patient does not have diabetes mellitus, cardiac disease, recent use of glucocorticoids, concurrent anticancer therapy, brain metastases, stroke, seizure disorder, active or uncontrolled infections, or other serious illnesses or medical conditions.

In some embodiments, the patient exhibits a biomarker of OSI-906 sensitivity or efficacy in ACC. In some embodiments, the patient selected overexpresses IGF2 gene transcripts. In some embodiments, the overexpression is at least about 10-fold, 25-fold, or 50-fold.

### COMPOUND AND FORMULATION

The active agent is OSI-906, which can be named as cis-3-[8-amino-1-(2-phenyl-quinolin-7-yl)-imidazo[1,5-a]pyrazin-3-yl]-1-methyl-cyclobutanol, or a pharmaceutically acceptable salt thereof. In some embodiments, the compound is in its free base form.

OSI-906 can be prepared and formulated according to U.S. 2006/0235031 or other suitable methods. In some embodiments, the OSI-906 is a formulated as an oral immediate release tablet, capsule, or the like, using conventional excipients.

### DOSING AND ADMINISTRATION

The invention provides a method of treating ACC comprising treating a patient in need thereof with an effective regimen comprising OSI-906 or a pharmaceutically acceptable salt thereof.

In some embodiments, the regimen is carried out without other anti-cancer agents.

In some embodiments, the OSI-906 is administered orally in an amount of about 0.5 to about 25 mg/kg·day, about 1 to about 15 mg/kg·day, about 2 to about 12 mg/kg·day, or about 4 to about 10 mg/kg·day on days of administration.

In some embodiments, the OSI-906 is administered only during the first 3, 4, 5, 6, or 7 days of each 14 day treatment period or cycle, wherein no drug is administered on the remaining days of each period. In some embodiments, the OSI-906 is administered every day.

In some embodiments, the regimen is continued until there is a significant adverse event, disease progression, patient request, or patient death.

### RESULTS

In some embodiments, the patient has stable ACC disease on the regimen for at least one half year, or for at least one year. In some embodiments, at least about 20% or at least about 40% of treated patients have stable ACC disease on the regimen for at least one half year, or for at least one year.

In some embodiments, the patient exhibits at least a partial response to the method as evaluated by RECIST. In some embodiments, the partel response as evaluated by RECIST is least about 30%, 40%, 50%, 60%, or 70%.

In some embodiments, the regimen does not result in drug-related toxicity.

### EXPERIMENTAL DATA

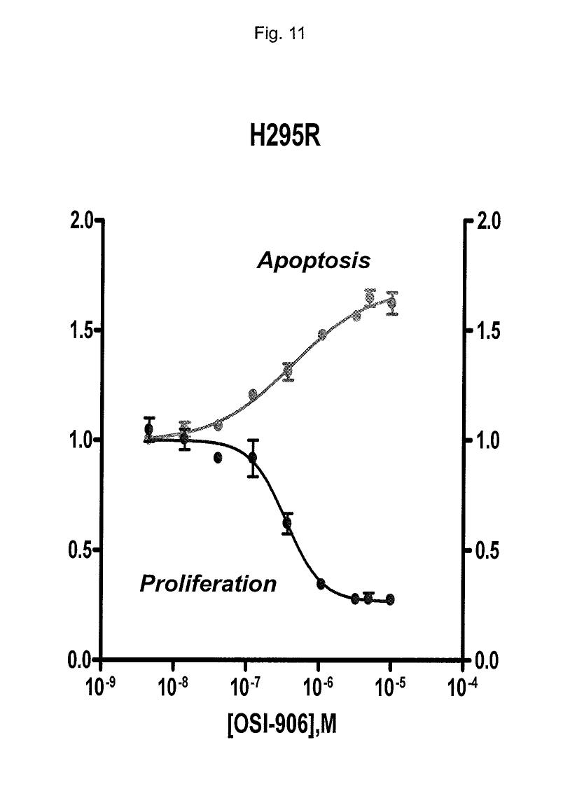

The H295R ACC tumor cell line exhibits sensitivity to OSI-906. H295R tumor cells were treated with varying concentrations of OSI-906, and measurements of proliferation (Cell Titer Glo, Promega) and apoptosis (Caspase Glo, Promega) were determined 72 and 48 hours after dosing, respectively. See FIG. 11.

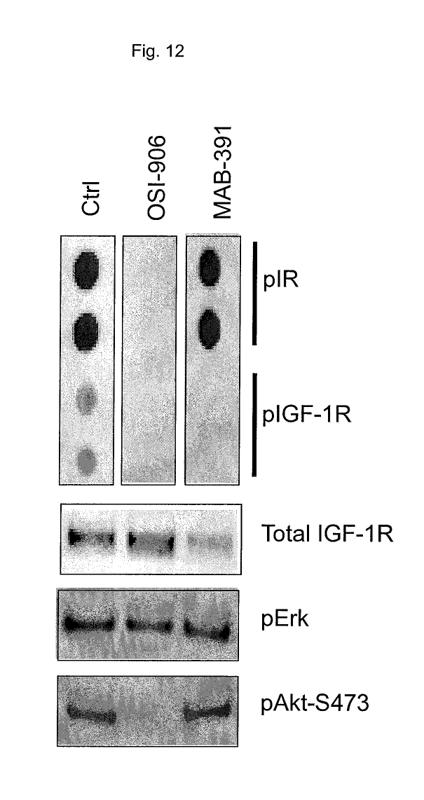

The H295R ACC tumor cell lines exhibits a high level of phosphorylated IGF-1 R and IR, and OSI-906 inhibits phosphorylation of both receptors, conferring inhibition of pAkt. H295R tumor cells were treated with 3 uM OSI-906 or 3 ug/ml of the IGF-1R neutralizing antibody MAB-391 for 24 hours. Measurement of pIGF-1R and pIR was determined by RTK capture array (ARY001, R&D Systems), and measurement of pAkt was determined by WB. See FIG. 12.

As of 14 Apr. 2009 in two phase I clinical trials, seven patients have been enrolled with ACC. One had partial response (Patient 1), two had stable disease for >6 months, and one had a dramatic, short-lived sympitomatic response, at three remain on study for 69+, 22+ and 8+ days.

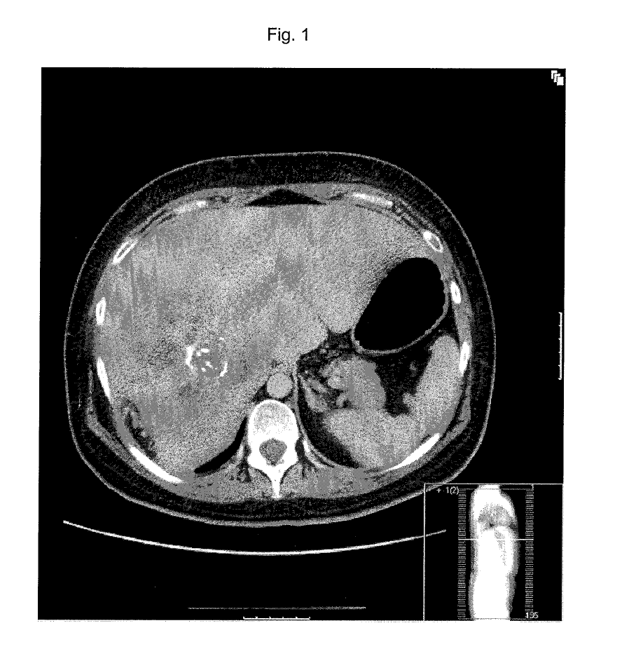

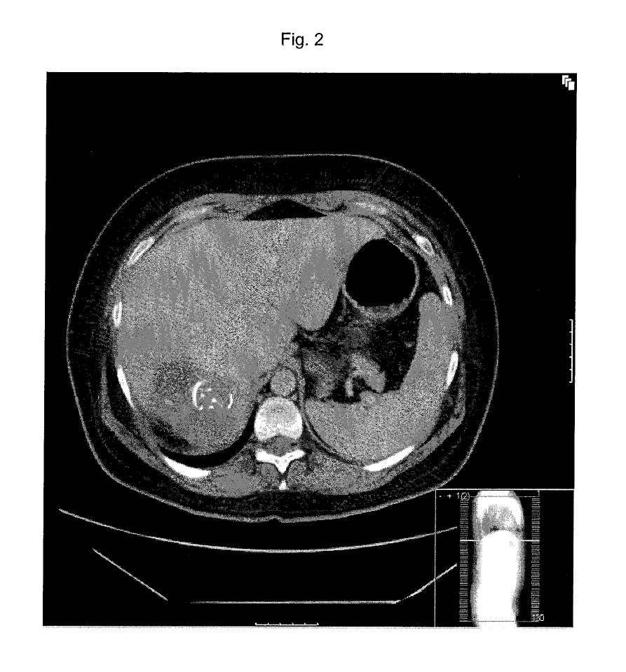

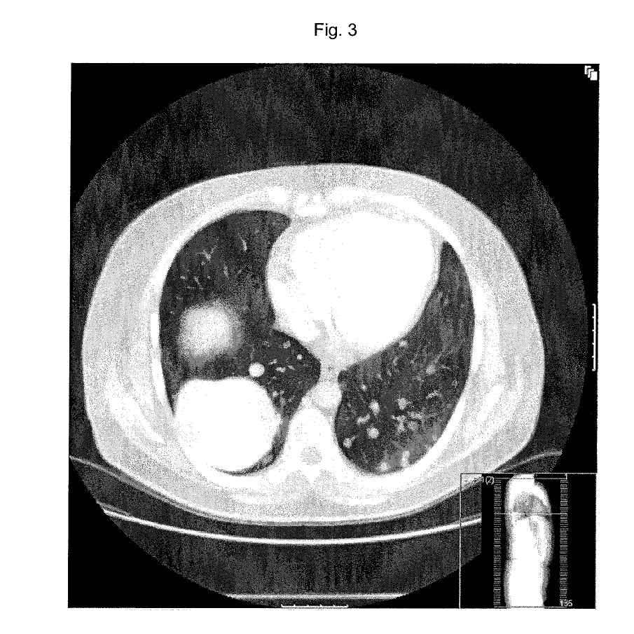

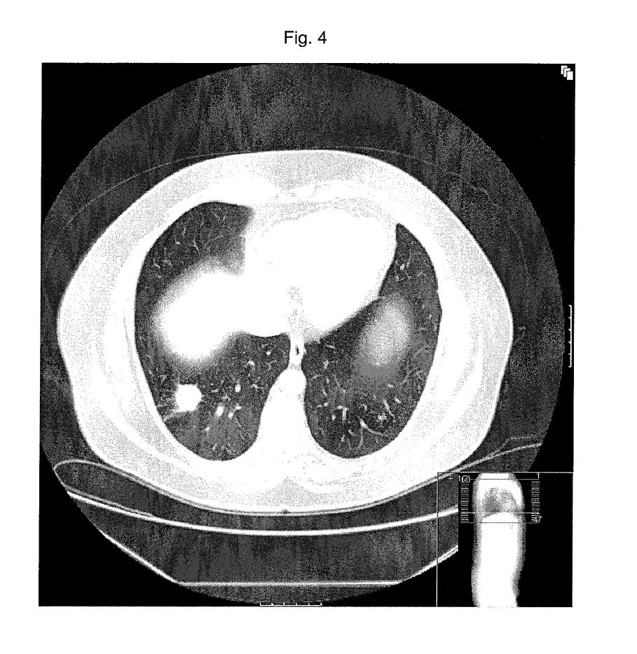

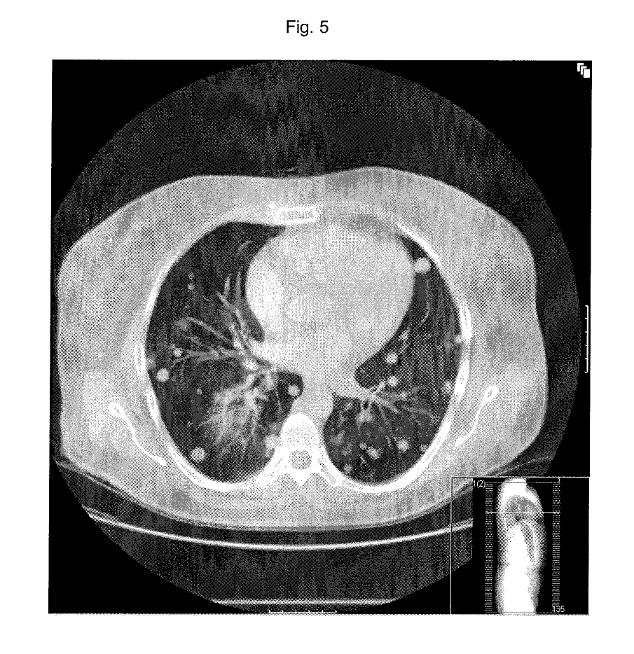

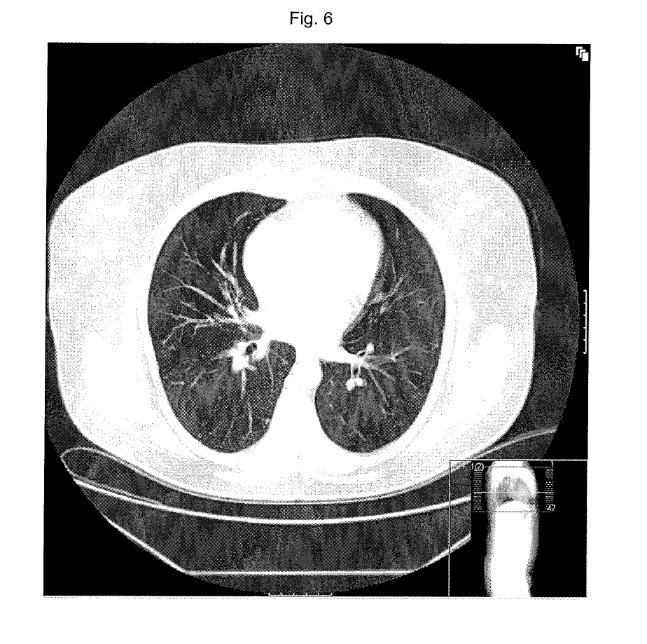

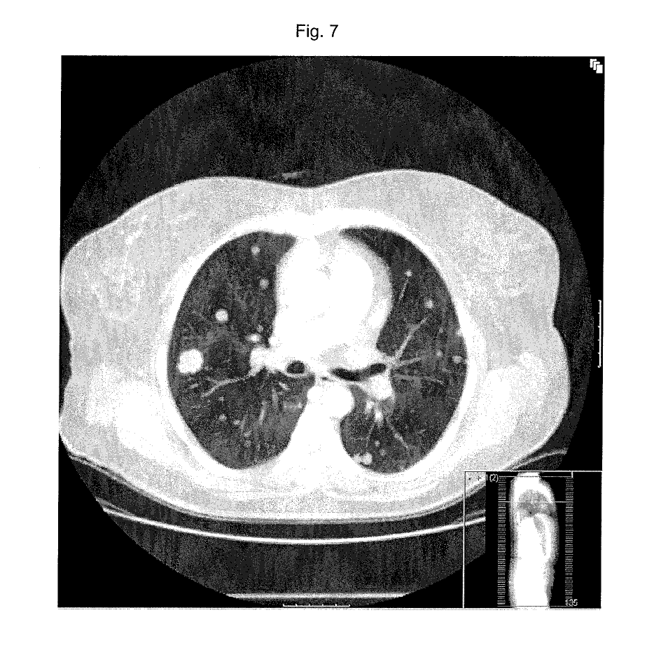

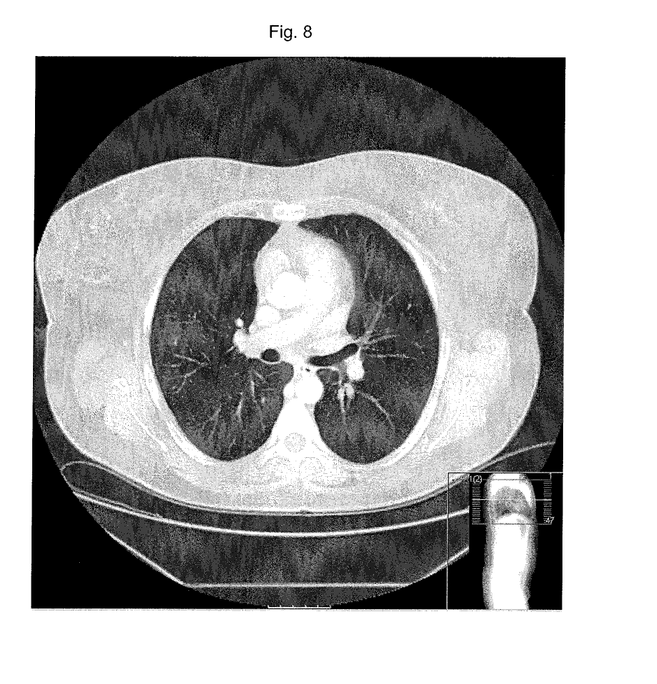

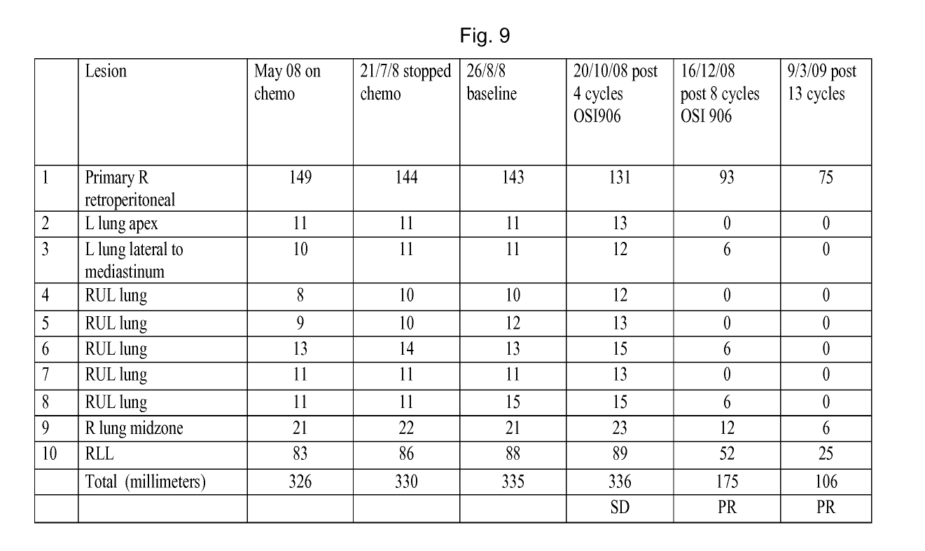

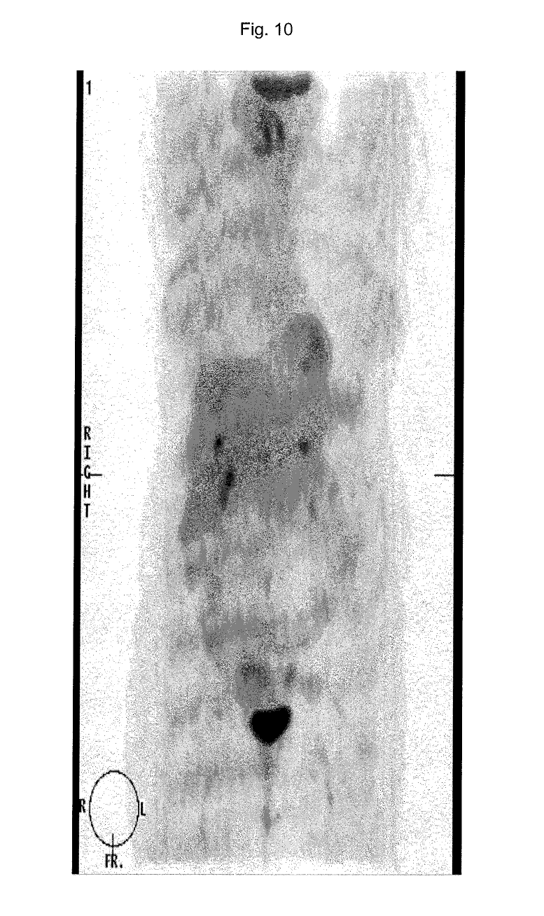

Patient 1 is a 35 year old female ACC patient, previously treated with six cycles of etoposide, cisplatin, and doxorubicin and mitotane from Feb. 2008 to Jul. 2008. The patient showed progressive disease. Patient 1 was then treated with OSI-906 beginning Sep. 1, 2008, 450 mg/day, escalating to 600 mg, on days 1-3 of each 14 day period. At 8 weeks of treatment, CT scan was reported as stable. At 16 weeks, CT scan showed a decrease in primary lesion as well as improvement in all pulmonary lesions. The overall reduction in RECIST measurement of target lesions was 43%. Subsequent scans indicated incremental response, including a scan showing a partial response of 72% decrease by RECIST compared to baseline. No drug-related toxicities have been observed up to 56 weeks. This patient had a 18-FDG-PET scan 30 weeks into treatment that showed no tumor uptake of the tracer in the residual primary tumor nor two of the remaining metastases visible on CT scan. ACC is typically thought to be an FDG-avid tumor. See FIGS. 1-10.

Objective Response Criteria (RECIST)

Complete Response: Disappearance of all clinical and radiological evidence of tumor (both target and nontarget) including normalization of elevated tumor markers at baseline, if documented. The patient must be free of all tumor-related symptoms. Complete Response must be confirmed at a second tumor assessment not less than 28 days apart from the assessment at which CR was observed.

Partial Response: At least a 30% decrease in the sum of Longest Diameter (LD) of target lesions taking as reference the baseline sum LD. Partial Response must be confirmed at a second tumor assessment not less than 28 days apart from the assessment at which PR was observed.

Stable Disease: Steady state of disease. Neither sufficient shrinkage to qualify for PR nor sufficient increase to qualify for progressive disease. Stable disease must be documented to be present at least 28 days from the start of the therapy. There may be no appearance of new lesions for this category.

Progressive Disease: At least a 20% increase in the sum of LD of measured lesions taking as references the smallest sum LD recorded since the treatment started. Appearance of new lesions will also constitute progressive disease. In exceptional circumstances, unequivocal progression of nontarget lesions may be accepted as evidence of disease progression (DP).

Tumor Measurement

All patients who have measurable disease according to RECIST, who received at least 2 treatment periods of therapy, and who have their disease re-evaluated will be evaluable for response. All sites of disease should be followed as either target or nontarget lesions, as categorized at baseline. All measurable lesions up to a maximum of 5 lesions per organ or 10 lesions in total, representative of all involved organs, should be identified as target lesions, while all other lesions (either additional measurable lesions or nonmeasurable lesions) should be classified as nontarget lesions. To ensure comparability, the baseline radiology/scans and subsequent radiology/scans to assess response should be performed using identical techniques (i.e., scans performed immediately following bolus contrast administration should be made with a standard volume of contrast, the identical contrast agent, and preferably the same scanner). The same method, radiological or physical, should be employed and assessed by the same individual on each occasion.

1. A method of treating adrenocortical carcinoma (ACC) comprising treating a patient in need thereof with an effective regimen comprising OSI-906 or a pharmaceutically acceptable salt thereof.

2. The method of claim 1, wherein the patient has not been previously treated for the ACC.

3. The method of claim 1, wherein the ACC is refractory to prior mitotane-containing treatment.

4. The method of claim 1, wherein the regimen is carried out without other anti-cancer agents.

5. The method of claim 1, wherein the regimen does not result in drug-related toxicity,

6. The method of claim 1, wherein the OSI-906 is administered orally in an amount of about 1 to about 15 mg/kg·day on days of administration.

7. The method of claim 1, wherein the OSI-906 is administered orally in an amount of about 2 to about 12 ring/kg·day on days of administration.

8. The method of claim 1, wherein the OSI-906 is administered on every day of the regimen.

9. The method of claim 1, wherein the OSI-906 is administered only during the first 3-7 days of each 14 day treatment period.

10. The method of claim 1, wherein the patient has stable ACC disease on the regimen for at least one half year.

11. The method of claim 1, wherein the patient has stable ACC disease on the regimen for at least one year.

12. The method of claim 1, wherein the patient exhibits at least a partial response to the method as evaluated by RECIST.

13. The method of claim 1, wherein the patient exhibits at least a partial response to the method as evaluated by RECIST in an amount of at least about 40%.

14. The method of claim 1, wherein the patient exhibits a biomarker of OSI-906 efficacy in ACC.

15. The method of claim 1, wherein the patient overexpresses IGF2 gene transcripts by at least about 10-fold.
